(19) United States
(12) Patent Application Publication
(10) Pub. No.: US 2006/0036373 A1
(43) Pub. Date: Feb. 16, 2006

(54) Method and system for cropping an image of a multi-pack of microarrays

(76) Inventors: Srinka Ghosh, San Francisco, CA (US); Peter G. Webb, Menlo Park, CA (US)

Correspondence Address: AGILENT TECHNOLOGIES, INC.;Legal Department, DL429, Intellectual Property Administration, P. O. Box 7599, Loveland, CO, 80537-0599, US

(21) Appl. No.: 10/915,849

(22) Filed: Aug. 11, 2004

(51) Int. Cl. G06F 19/00; G06K 9/00; G01N 33/48; G01N 33/50

(52) U.S. Cl. 702/20; 382/128

(57) ABSTRACT

A method and system for cropping a digital image of multiple individual microarrays. Various embodiments of the present invention include, a digital image of multiple individual microarrays projected along a first coordinate axis by summing columns of pixel intensity values. A transformation maps the projected pixel intensity values to a transform in a frequency domain. A filter function is constructed from a power spectrum of the transform and multiplied by the transform to obtain a filtered transform. The filtered transform is mapped back to the spatial domain to give the filtered, spatial-domain image. The filtered, spatial-domain image is used to determine the coordinates of boundaries separating the individual microarrays along the first coordinate axis. The multi-pack of microarrays is rotated, and the method may be repeated for a second coordinate axis that is perpendicular to the first coordinate axis. The boundaries are used to identify the boundaries separating individual microarrays.

Embodiments of the present invention are related to extracting data from images of microarrays and, in particular, to a method and system for cropping an image of a multi-pack of microarrays.

### BACKGROUND OF THE INVENTION

The present invention is related to microarrays. In order to facilitate discussion of the present invention, a general background for microarrays is provided below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques to analyze complex solutions of any type of molecule that can be optically or radiometrically scanned and that can bind with high specificity to complementary molecules synthesized within, or bound to, discrete features on the surface of a microarray. Because microarrays are widely used for analysis of nucleic acid samples, the following background information on microarrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. Phosphorylated subunits of DNA and RNA molecules, called “nucleotides,” are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations A, T, C, and G for the nucleotide subunits that together compose the DNA polymer. For example, the oligomer 100 shown in FIG. 1 can be chemically represented as “ATCG.”

The DNA polymers that contain the organization information for living organisms occur in the nuclei of cells in pairs, forming double-stranded DNA helices. One polymer of the pair is laid out in a 5′ to 3′ direction, and the other polymer of the pair is laid out in a 3′ to 5′ direction, or, in other words, the two strands are anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

FIGS. 4-7 illustrate the principle of microarray-based hybridization assays. A microarray (402 in FIG. 4) comprises a substrate upon which a regular pattern of features is prepared by various manufacturing processes. The microarray 402 in FIG. 4, and in subsequent FIGS. 5-7, has a grid-like 2-dimensional pattern of square features, such as feature 404 shown in the upper left-hand corner of the microarray. Each feature of the microarray contains a large number of identical oligonucleotides covalently bound to the surface of the feature. These bound oligonucleotides are known as probes. In general, chemically distinct probes are bound to the different features of a microarray, so that each feature corresponds to a particular nucleotide sequence.

Once a microarray has been prepared, the microarray may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the microarray. FIG. 5 shows a number of such target molecules 502-504 hybridized to complementary probes 505-507, which are in turn bound to the surface of the microarray 402. Targets, such as labeled DNA molecules 508 and 509, that do not contain nucleotide sequences complementary to any of the probes bound to the microarray surface do not hybridize to generate stable duplexes and, as a result, tend to remain in solution. The sample solution is then rinsed from the surface of the microarray, washing away any unbound-labeled DNA molecules. In other embodiments, unlabeled target sample is allowed to hybridize with the microarray first. Typically, such a target sample has been modified with a chemical moiety that will react with a second chemical moiety in subsequent steps. Then, either before or after a wash step, a solution containing the second chemical moiety bound to a label is reacted with the target on the microarray. After washing, the microarray is ready for analysis. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the microarray as shown in FIG. 7, with features to which labeled target molecules are hybridized similar to 702 optically or digitally differentiated from those features to which no labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the microarray was exposed, of labeled DNA complementary to the oligonucleotide within the feature.

A multiple of individual microarrays, such as those described above with reference to FIGS. 4-7, can be arranged on a single slide or substrate to form a multi-pack of microarrays. FIG. 8 is an illustration of an example 8-pack of microarrays 802 having eight microarrays 804-811. In FIG. 8, vertical and horizontal dashed lines 812-815 are the boundaries that indicate the separation between the individual microarrays within the multi-pack of microarrays 802. Generally, knowledge of the locations and orientation of the individual microarrays allows for further analysis of any one particular microarray within a multi-pack of microarrays. Therefore, designers, manufacturers, and users of microarrays and microarray readers seek computationally efficient methods for cropping an image of a multi-pack of microarrays.

### SUMMARY OF THE INVENTION

One of various embodiments of the present invention comprises a method and system for cropping a digital image of multiple individual microarrays. Various embodiments of the present invention include projecting the digital image along a first coordinate axis by summing columns of pixel intensity values to form a spatial-domain image. A transformation is employed to map the spatial-domain image to a transform in a frequency domain. A power spectrum of the transform is computed and used to determine a filter function. The filter function is multiplied by the transform leaving the transform of the individual microarray boundaries. An inverse transform is employed to map the filtered transform into a filtered, spatial-domain image. The filtered, spatial-domain image is used to determine the locations of the boundaries of the individual microarrays along the first coordinate axis. The digital image of the multi-pack of microarrays may be rotated and the method can be repeated for a second coordinate axis. The boundaries are used to identify the boundaries separating the individual microarrays.

### BRIEF DESCRIPTION OF THE DRAWINGS

FIG. 1 illustrates a short DNA polymer.

FIGS. 2A-B illustrate the hydrogen bonding between the purine and pyrimidine bases of two anti-parallel DNA strands.

FIG. 3 illustrates a short section of a DNA double helix comprising a first strand and a second, anti-parallel strand.

FIG. 4 illustrates a grid-like, two-dimensional pattern of square features.

FIG. 5 shows a number of target molecules hybridized to complementary probes, which are in turn bound to the surface of the microarray.

FIG. 6 illustrates the bound labeled DNA molecules detected via optical or radiometric scanning.

FIG. 7 illustrates optical, radiometric, or other types of scanning produced by an analog or digital representation of the microarray.

FIG. 8 is an illustration of eight microarrays arranged on a single slide to form an 8-pack microarray.

FIG. 9 is a three-dimensional depiction of a two-dimensional pixel image matrix I(x,y).

FIG. 10 shows an N×M pixel image matrix representing the digital image of a multi-pack of microarrays.

FIG. 11 illustrates a rotational discrepancy between orientations of coordinate axes of the 8-pack of microarrays shown in FIG. 8 and orientations assumed by a microarray reader.

FIG. 12 is a control-flow diagram of a method for cropping a multi-pack of microarrays that represents one of many possible embodiments of the present invention.

FIG. 13 is an example image of an 8-pack of microarrays.

FIG. 14 shows a 6720×2160 pixel image matrix I(x,y) for the 8-pack of microarrays shown in FIG. 13.

FIGS. 15A-B show sampled pixel image matrices that result from convolving the pixel image matrix I(x,y) shown in FIG. 14 with a sampling function s(x,y).

FIG. 16 illustrates projecting pixel intensities along the x coordinate axis of the pixel digital image of a 4-pack of microarrays.

FIG. 17 illustrates numerical calculation of a portion of a projection corresponding to a single, four-feature microarray of a multi-pack of microarrays.

FIG. 18 illustrates projecting the sampled spatial-domain image of the example 8-pack of microarrays shown in FIG. 15 along the x coordinate axis.

FIG. 19 is a diagram of a cropping method that represents one of many possible embodiments of the present invention.

FIG. 20 displays Fourier transform elements and the interpretation of each element.

FIGS. 21A-B shows a power spectrum computed for the spatial-domain image f(x) shown in FIG. 18.

FIG. 22 shows a top-hat, bandpass filter function.

FIGS. 23A-B show a filtered, spatial-domain image g(x) corresponding to the un-filtered, spatial-domain image j(x) shown in FIG. 18.

FIG. 24A-B are illustrations of a peak envelope of the filtered projection g(x) shown in FIG. 23B.

FIGS. 25A-C display re-scaled x coordinates of the microarray edges of the example 8-pack of microarrays.

FIG. 26 illustrates projecting the sampled spatial-domain image of the example 8-pack of microarrays, shown in FIG. 13, after rotating the spatial domain by 90 degrees.

FIG. 27 shows boundaries of the 8-pack of microarrays shown in FIG. 13.

FIG. 28 is a control-flow diagram for the routine “auto-cropping a multi-pack of microarrays.”

FIG. 29 is a control-flow diagram for the routine “determine a number of points required for FFT.”

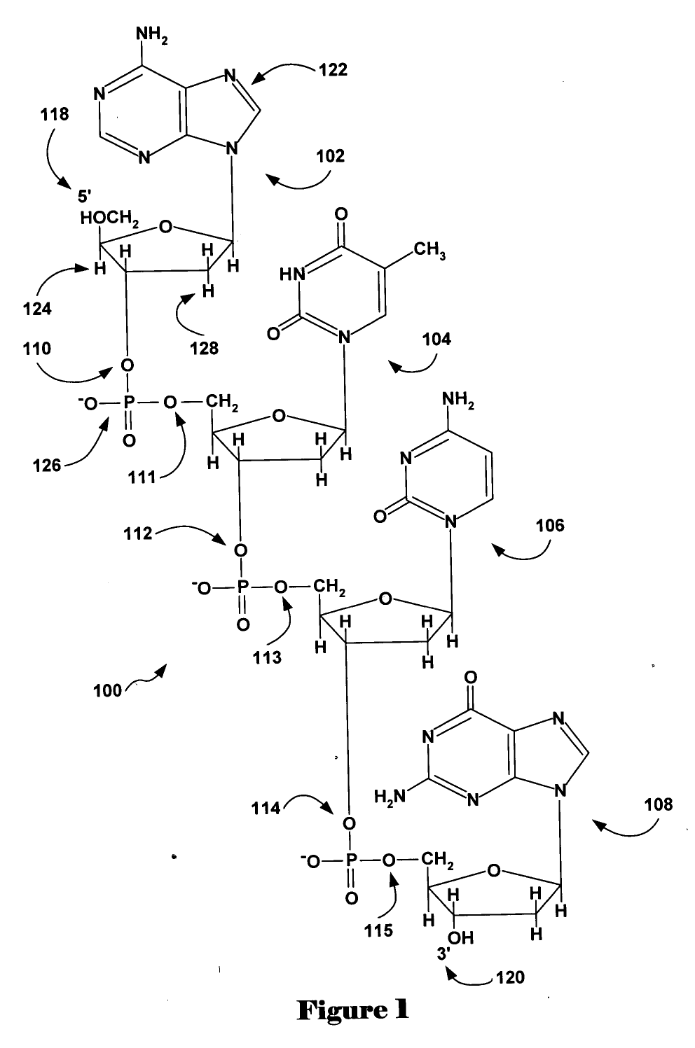

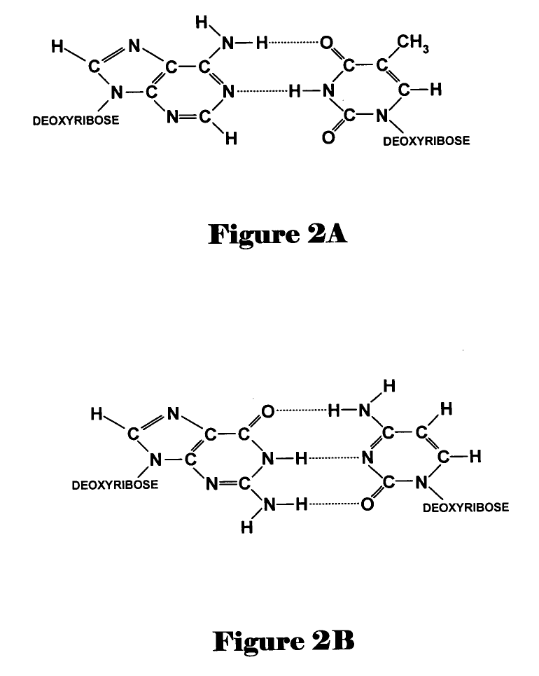

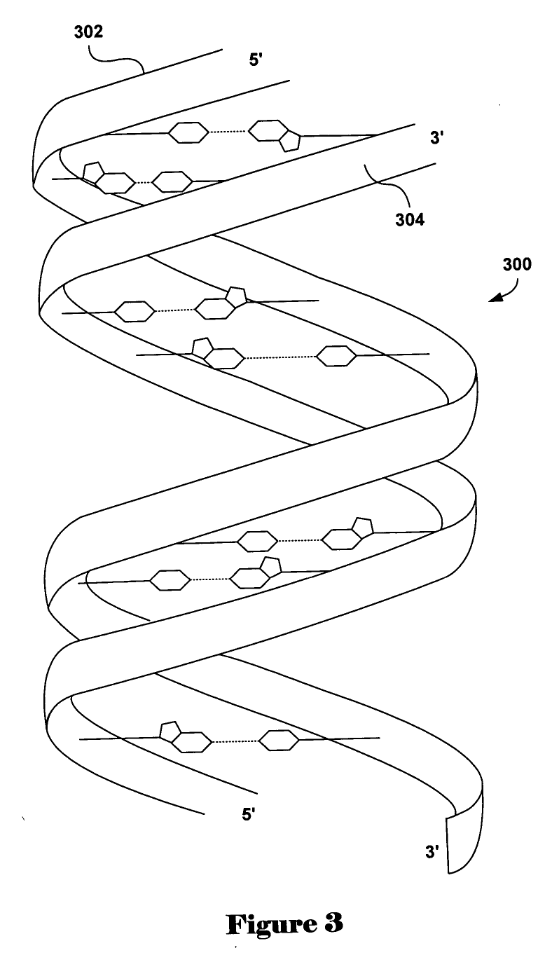

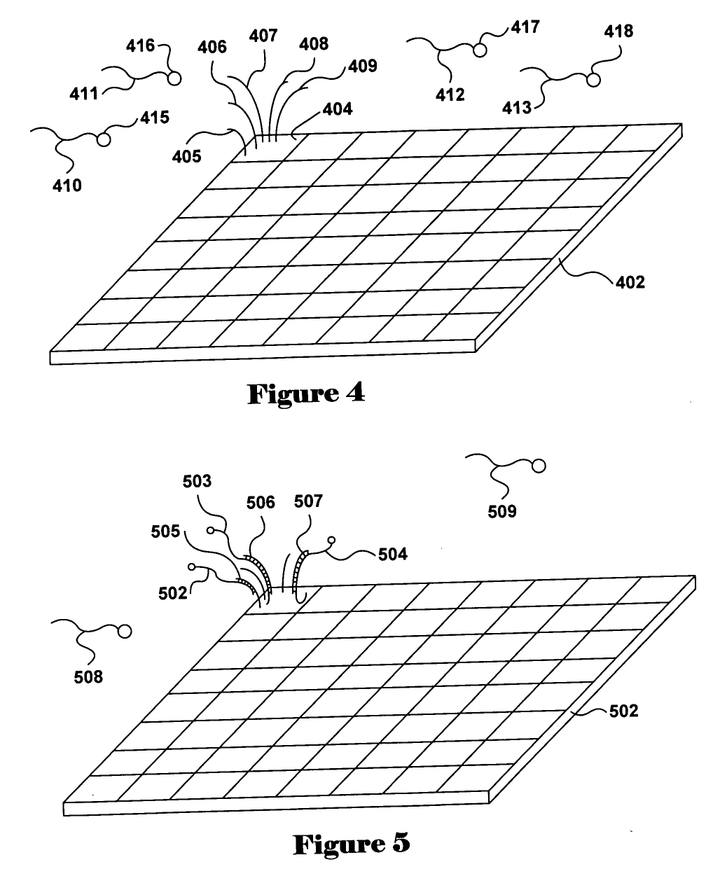

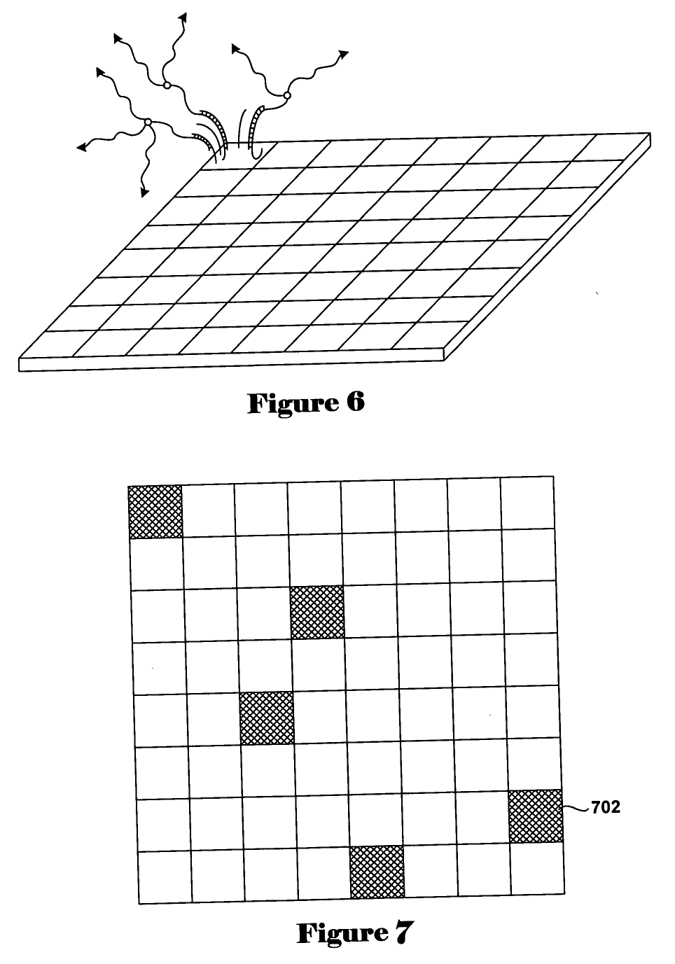

### DETAILED DESCRIPTION OF THE INVENTION

The present invention is directed toward an automated method and system for cropping an image of a multi-pack of microarrays. Various embodiments of the present invention include software programs running on a single-processor computer system, or running in parallel, on multi-processor computer systems, or a larger number of distributed, interconnected single-and/or-multiple processor computer systems, or implemented directly in firmware or a combination of firmware and hardware. The present invention is described, in part below, with reference to a concrete problem, and with reference to graphical illustrations, control-flow diagrams, and mathematical equations, and includes the following four subsections: (1) Additional Information about Microarrays; (2) Additional Information about Multi-pack Microarrays; (3) Cropping a Multi-Pack of Microarrays; and (4) Implementation.

### Additional Information About Microarrays

A microarray may include any one-, two- or three-dimensional arrangement of addressable regions, or features, each bearing a particular chemical moiety or moieties, such as biopolymers, associated with that region. Any given microarray substrate may carry one, two, or four or more microarrays disposed on a front surface of the substrate. Depending upon the use, any or all of the microarrays may be the same or different from one another and each may contain multiple spots or features. A typical microarray may contain more than ten, more than one hundred, more than one thousand, more ten thousand features, or even more than one hundred thousand features, in an area of less than 20 cm² or even less than 10 cm^{2.} For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the microarrays are formed by processes involving drop deposition of reagents, but may not be present when, for example, photolithographic microarray fabrication processes are used. When present, interfeature areas can be of various sizes and configurations.

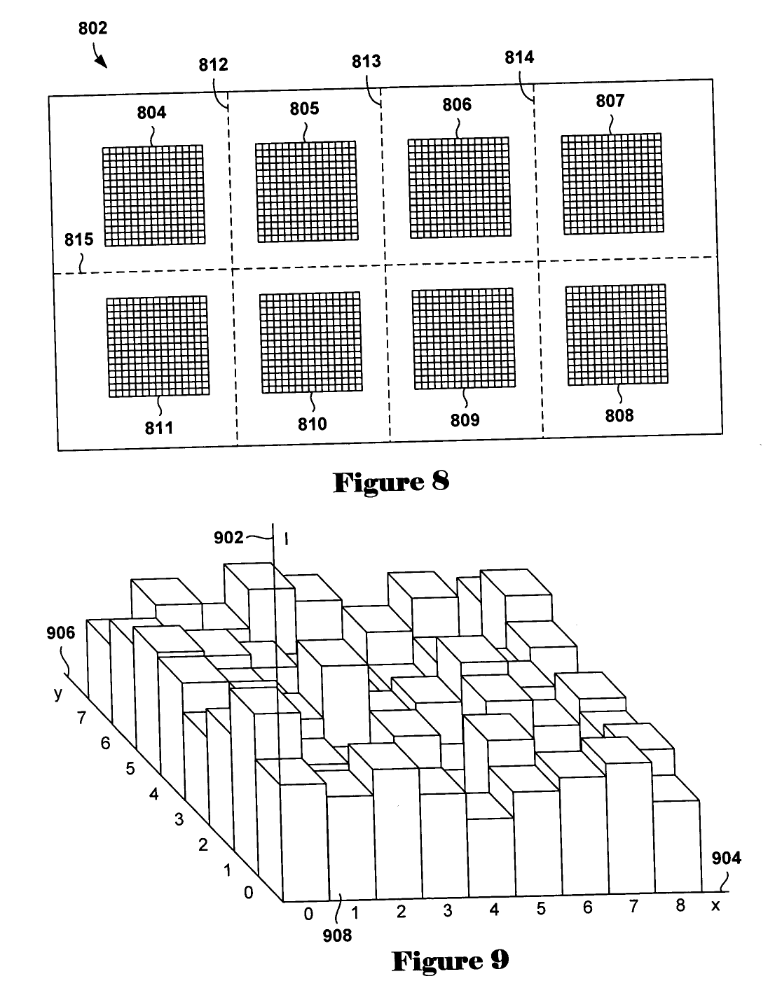

Each microarray may cover an area of less than 100 cm², or even less than 50 cm², 10 cm² or 1 cm². In many embodiments, the substrate carrying the one or more microarrays (see e.g., FIG. 8) will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With microarrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Microarrays can be fabricated using drop deposition from pulsejets of either polynucleotide precursor units (such as monomers) in the case of in situ fabrication, or the previously obtained polynucleotide. Such methods are described in detail in, for example, U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic microarray fabrication methods may be used. Interfeature areas need not be present particularly when the microarrays are made by photolithographic methods.

A microarray is typically exposed to a sample including labeled target molecules, or, as mentioned above, to a sample including unlabeled target molecules followed by exposure to labeled molecules that bind to unlabeled target molecules bound to the microarray, and the microarray is then read. Reading of the microarray may be accomplished by illuminating the microarray and reading the location and intensity of resulting fluorescence at multiple regions on each feature of the microarray. For example, a scanner may be used for this purpose, which is similar to the AGILENT MICROARRAY SCANNER manufactured by Agilent Technologies, Palo Alto, Calif. Other suitable apparatus and methods are described in published U.S. patent applications 20030160183A1, 20020160369A1, 20040023224A1, and 20040021055A, as well as U.S. Pat. No. 6,406,849. However, microarrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, and elsewhere.

A result obtained from reading a microarray, followed by application of a method of the present invention, may be used in that form or may be further processed to generate a result such as that obtained by forming conclusions based on the pattern read from the microarray, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically tran-sporting that item or, in the case of data, physically transporting a medium carrying the data or communicating the data.

As pointed out above, microarray-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the microarray that would bind to soluble labeled antigens in a sample solution. Many other types of chemical assays may be facilitated by microarray technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for microarray-based analysis. A fundamental principle upon which microarrays are based is that of specific recognition, by probe molecules affixed to the microarray, of target molecules, whether by sequence-mediated binding affinities, binding affinities based on conformational or topological properties of probe and target molecules, or binding affinities based on spatial distribution of electrical charge on the surfaces of target and probe molecules.

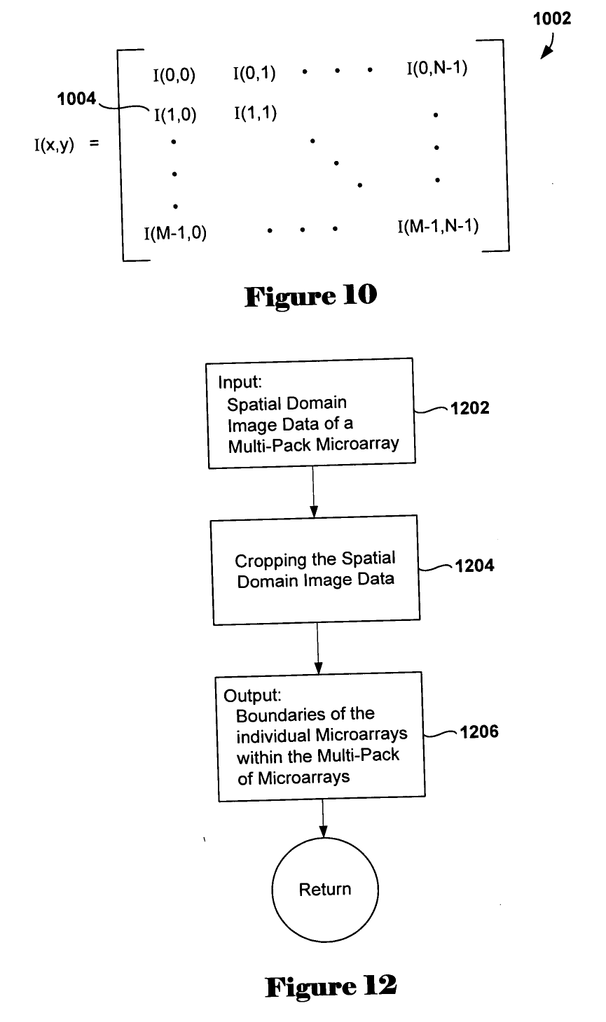

As described above with reference to FIGS. 9-10, scanning of a microarray by an optical scanning device or radiometric scanning device generally produces an image comprising a rectilinear grid of pixels, with each pixel having a corresponding signal intensity. These signal intensities are processed by a microarray-data-processing program that analyzes data scanned from an microarray to produce experimental or diagnostic results which are stored in a computer-readable medium, transferred to an intercommunicating entity via electronic signals, printed in a human-readable format, or otherwise made available for further use. Microarray experiments can indicate precise gene-expression responses of organisms to drugs, other chemical and biological substances, environmental factors, and other effects. Microarray experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Additional Information about Multi-pack Microarrays

When a multi-pack of microarrays is analyzed, data may be collected as a two-dimensional digital image of the multi-pack of microarrays, each pixel of which represents the intensity of phosphorescent, fluorescent, chemiluminescent, or radioactive emission from an area of the multi-pack of microarrays corresponding to the pixel. The digital image data set of a multi-pack of microarrays may comprise a two-dimensional image or a list of numerical or alphanumerical pixel intensities, or any of many other computer-readable data sets.

An initial series of steps employed in processing the digital image of the multi-pack of microarrays includes constructing a regular coordinate system for describing the location of each pixel. FIG. 9 is a three-dimensional graphical illustration of an 8-pixel×7-pixel sub-image from an N-pixel×M-pixel digital image of a multi-pack of microarrays. In FIG. 9, pixel values are shown by the height of the columns ascending vertically above a two-dimensional plane, where each pixel is plotted with respect to an intensity-axis 902 and positional axes comprising an x-axis 904 and y-axis 906. Each pixel value may be an 8-bit, 16-bit, or larger bytes that corresponds to the measured intensity of light emitted from a corresponding region of the multi-pack of microarrays surface. The positional axes 904 and 906 provide a regular coordinate system, referred to as the “pixel-coordinate domain,” used to describe the location of each pixel. For example, the location of pixel 908 can be specified by the pixel coordinates (1,0).

FIG. 9 is the equivalent of a three-dimensional depiction of a two-dimensional pixel image matrix denoted by I(x,y), where each element of the image matrix represents the digitized, grayscale, pixel intensities at the spatial domain coordinates (x,y). FIG. 10 shows the pixel image matrix 1002 that represents the N×M digital image of the multi-pack of microarrays described above in relation to FIG. 9, where N is the number of pixels along the x-axis 904, and M is the number of pixels along the y-axis 906. For example, pixel 1004 of the image matrix 1002 represents the pixel 908 shown in FIG. 9.

In general, each pixel of a multi-pack of microarrays is the sum of: (1) a signal-intensity component produced, at a location of the surface of the microarray corresponding to the pixel, by bound target molecules; and (2) a background-intensity component produced by a wide variety of background-intensity-producing sources, including noise produced by electronic and optical components of a microarray analysis instrument, general non-specific reflection of light from the surface of the microarray during scanning, or, in the case of radio-labeled target molecules, natural sources of background radiation, and various defects and contaminants on, and damage associated with, the surface of the microarray.

After the digital image data of the multi-pack of microarrays has been collected, cropping is employed to determine the locations and orientations of the individual microarrays within the multi-pack of microarrays. Typically, manual cropping is employed to crop the digital image. However, the cropped image and the original image are typically resaved, causing an increased demand for data storage. Cropping images of multi-packs of microarrays may also be accomplished by employing a microarray-design-layout file based on the expected printing locations of the microarrays as well as the number of microarrays expected per layout. However, on occasion, the microarray-design-layout file may not allow for variations that might occur during the actual process of printing the microarray. In certain cases, determination of the individuation microarray locations and orientations within the multi-pack of microarrays may be further complicated by a rotational discrepancy between the orientation of the rectilinear grid of pixels and the horizontal and vertical axes of the microarray reader. FIG. 11 illustrates the rotational discrepancy between the pixel coordinate axes of the 8-pack of microarrays shown in FIG. 8 and the orientations assumed by a microarray reader. In FIG. 11, the pixel coordinate axes 1102 and 1104 of the 8-pack of microarrays 1102 are rotated by “θ” degrees with respect to the coordinate axes 1108 and 1110 assumed by the microarray reader to correspond to the orientation of the multi-pack of microarrays region 1112.

### Cropping a Multi-pack of Microarrays

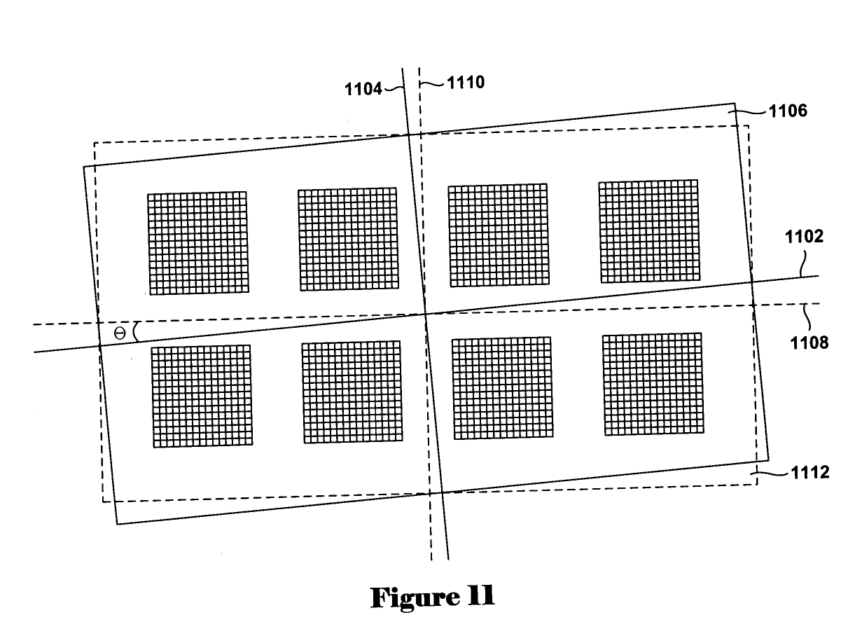

The method of the present invention can be applied to a spatial-domain image of a multi-pack of microarrays in which the orientation of the coordinate axes of the microarray is rotated, skewed, or stretched with respect to the image axes. FIG. 12 is a control-flow diagram of a method for cropping a digital image of a multi-pack of microarrays that represents one of many possible embodiments of the present invention. In step 1202, spatial-domain image data of a multi-pack of microarrays is received. The spatial-domain image data may be stored as a digital file residing in the memory or storage medium to which the file has been transferred (for example, a hard drive or CDROM). Next, in step 1204, one of many possible embodiments of the present invention is employed to crop the spatial-domain image data of the multi-pack of microarrays. Finally, in step 1206, indications of the locations and orientations of the one or more individual microarray boundaries within the multi-pack of microarrays is output.

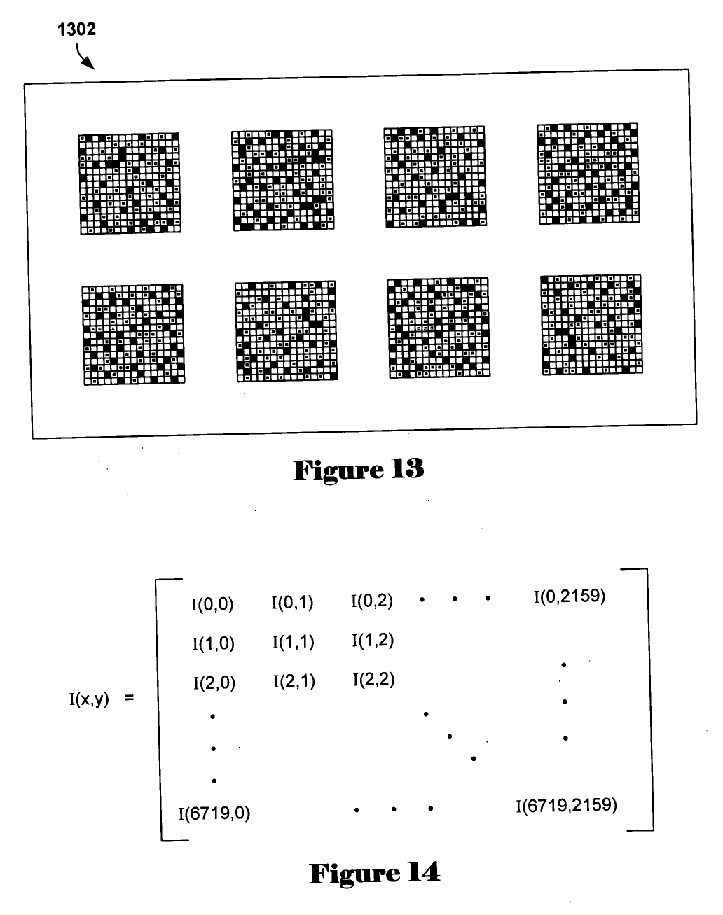

One of many possible embodiments of the method of the present invention is applied to the digital image data of an example image of an 8-pack of microarrays. Note that the present invention is not limited to the multipack of microarrays shown in FIG. 8. The present invention can be employed for any number of possible arrangements of multiple individual microarrays. FIG. 13 is an example image of an 8-pack of microarrays 1302. The spatial domain of the 8-pack of microarrays has 6720×2160 (N×M) or 14,515,200 pixels. FIG. 14 shows the 6720×2160 pixel image matrix I(x,y) for the example 8-pack of microarrays shown in FIG. 13. For an 8-pack of microarrays having 6720×2160 pixels, where each pixel representing 64 (2⁶) different intensity levels, more than 87,091,200 (6720×2160×6) bits are needed to store the entire digital image. Therefore, the spatial domain is typically sampled in order to increase the computational efficiency of the cropping method of the present invention by decreasing the amount of the digital image data.

Sampling the 6720×2160 digital image of the 8-pack of microarrays shown in FIGS. 13 and 14 can be formulated mathematically by convolving the pixel image matrix I(x,y) with a sampling function referred to as “s(x,y).” One of many possible sampling functions s(x,y) used to sample the pixel image matrix I(x,y) may be mathematically characterized by the following equation:

$$s ( x , y ) = \{ \begin{matrix}1 & if x = nX and y = mY \\ 0 & otherwise\end{matrix} \qquad Equation ( 1 )$$

where X and Y are integers;

n is an integer ranging from 0,1,2, . . . ,

$${N}_{1} = ( \frac{N}{X} - 1 ) ;$$

m is an integer ranging from 0,1,2, . . . ,

$${M}_{1} = ( \frac{M}{Y} - 1 ) ;$$

and

N₁ and M₁ are integers.
Convolving the digital image I(x,y) with the sampling function s(x,y) can be characterized by the following expression:

$$I ( x , y ) * ( x , y ) = {\sum }_{x = 0}^{N} {\sum }_{y = 0}^{M} I ( x , y ) s ( x , y ) \\ = I ( nX , nY ) \qquad Equation ( 2 )$$

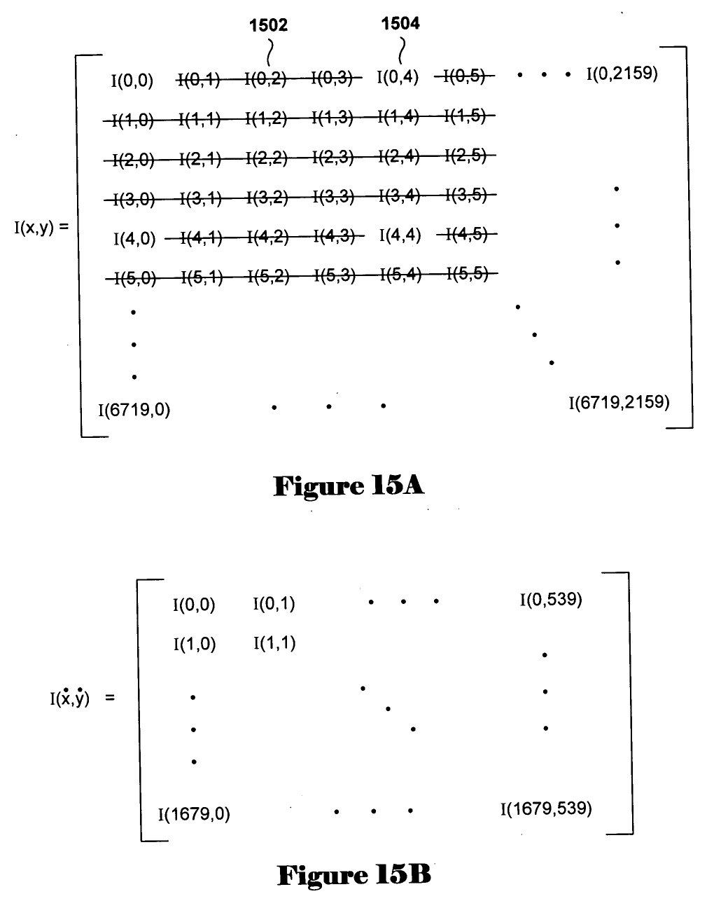

FIGS. 15A-B show the sampled pixel image matrices that result from convolving the pixel image matrix I(x,y) shown in FIG. 14 with the sampling function s(x,y), where both X and Y are assigned the value “4.” In FIG. 15A, the pixels eliminated from the pixel image matrix are identified by lines drawn through the image element, such as pixel 1502, and the sampled image elements are left unchanged, such as pixel 1504. Sampling the pixel image matrix I(x,y) of the 8-pack of microarrays according to equation (1), where X and Y equal “4,” reduces the original 6720×2160 spatial domain to a 1680×540 spatial domain. In FIG. 15B, the spatial domain has been re-indexed to provide a compact monotonically increasing index along the x and y directions.

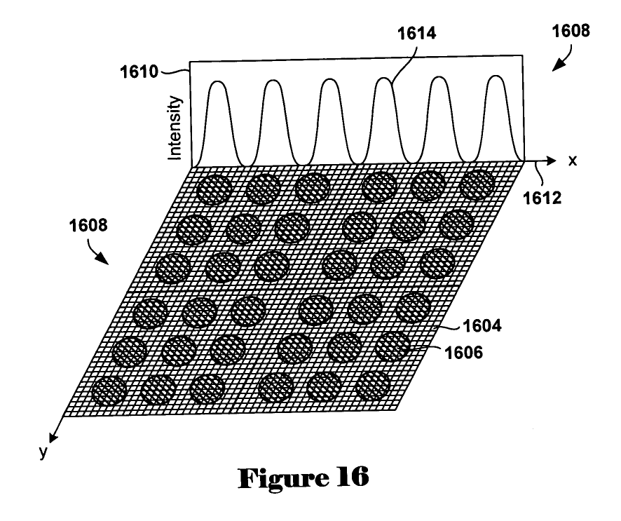

After the digital image data has been sampled, the pixel intensities are projected along the x or y coordinate axis. FIG. 16 illustrates projecting the pixel intensities along the x coordinate axis of a hypothetical pixel digital image of a 4-pack of microarrays. The image of a hypothetical microarray 1602 is represented in FIG. 16 as a grid of pixels 1604, with the higher intensity pixels corresponding to features illustrated as dark circles, such as the disk-shaped group of pixels 1606. The intensity levels of the pixels are projected along the x-axis to produce a projection 1608. The projection 1608 is illustrated as a two-dimensional graph, where the total projected intensity value is plotted in intensity axis 1610 with respect to the x coordinate axis 1612. Projection of the intensity values produces a wave-like graph 1614.

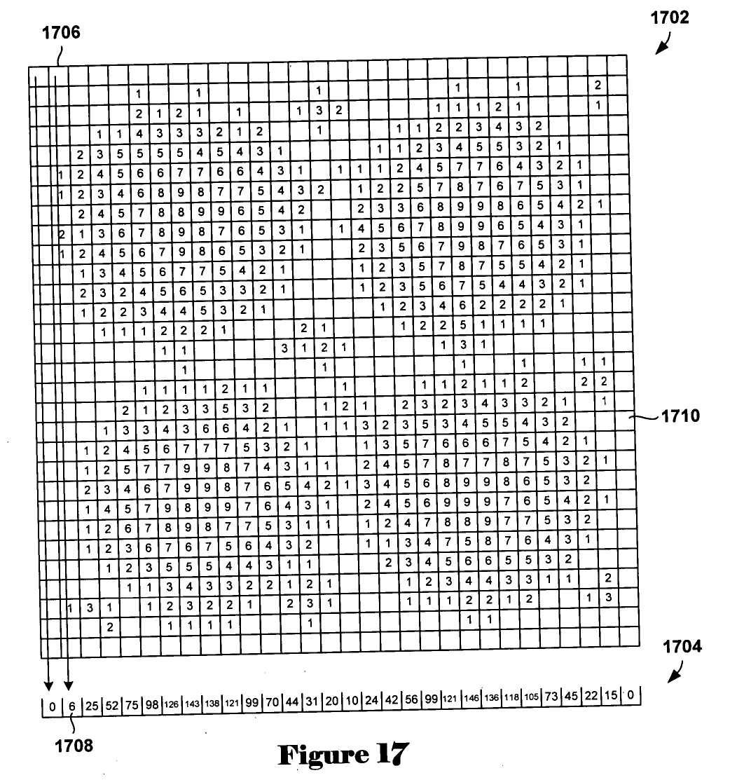

FIG. 17 illustrates numerical calculation of a portion of a projection corresponding to a single, four-feature microarray of a multi-pack of microarrays. A projection is calculated for all features, as described in the above paragraph, and contains a number of peaks. However, for the sake of simplicity of illustration, FIG. 17 shows pixel intensity values for a single, four-feature microarray, and the method of projecting will therefore produce two peaks. The intensity levels of all the pixels in each column of the grid of pixels 1702 are summed, and the sums are entered into the linear array 1704. For example, column 1706 includes five non-zero pixels having intensity values 1, 1, 2, 1, and 1. Thus, summing all the intensity values of the pixels in column 1706 produces the sum 6 (1708 in FIG. 17) in the second element of array 1704 corresponding to column 1706. Note that, in FIG. 17, “0” intensity values are not explicitly shown, and pixels having intensity value of “0” are shown as blank, or unfilled, squares, such as pixel 1710. Note also that other operations, such as averaging, may be performed as an alternative to summing columns of pixels to create a projection.

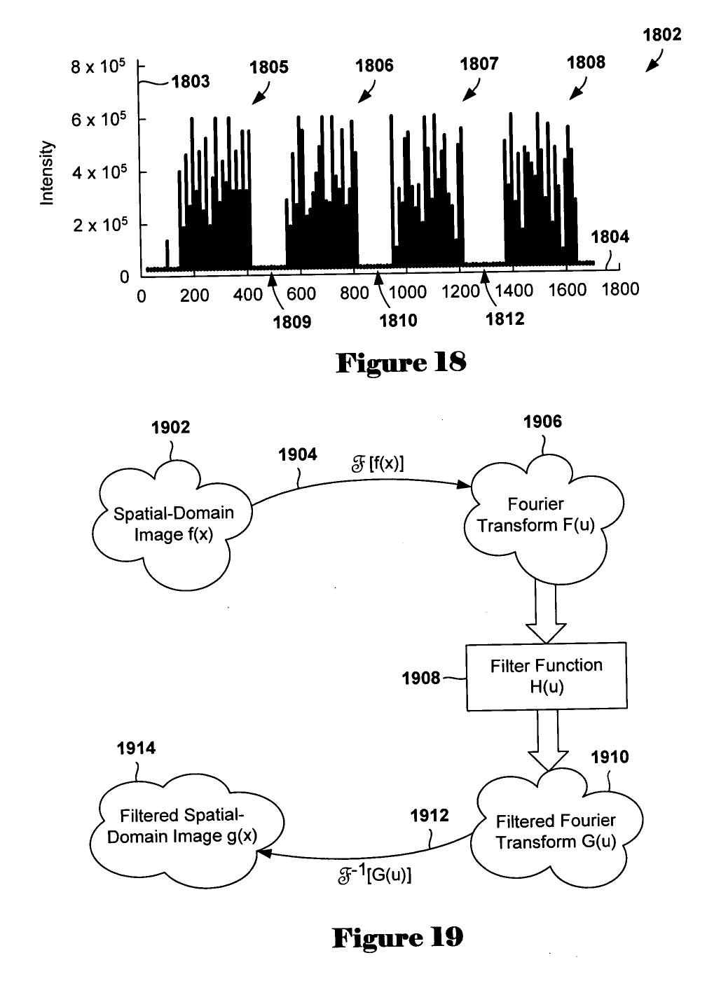

FIG. 18 shows the projection 1802 resulting from projecting the sampled digital image of the example 8-pack of microarrays shown in FIG. 15 along the x coordinate axis. Projection 1802 is plotted with respect to the intensity-axis 1808 and the x-axis 1810. Projection 1802 has four intensity bands 1803-1806, and troughs 1808-1810 that correspond to the sum of pixel intensities between microarrays. The irregular intensities within each intensity band 1803-1806 are the result of the rotated or skewed orientation of the 8-pack of microarrays. The projection 1802 is referred to as the “spatial-domain image,” denoted by “f(x),” the x-axis 1810 is referred to as the “one-dimensional spatial domain” or “spatial domain,” and values in the spatial domain are referred to as “points.”

The Fourier transformation method is based on a mathematical theorem, which states that it is possible to represent any function as a summation of a series of sine and cosine functions, each having a different combination of frequency, amplitude, and phase. FIG. 19 is a diagram of a cropping method that represents one of many possible embodiments of the present invention. The discrete Fourier transform is a one-to-one mapping from the spatial-domain image f(x) 1902 to the frequency domain, denoted [symbol] [f(x)] 1904, and is defined by the following equation:

$$ℱ [ f ( x ) ] = F ( u ) = \frac{1}{N} {\sum }_{x = 0}^{{N}_{1} - 1} f ( x ) exp ( \frac{- 2 π xui}{{N}_{1}} ) \qquad Equation ( 3 )$$

where i=\sqrt{−1};

N₁=the number of points in the spatial domain x; and

F(u) 1906 is referred to as the “Fourier transform.”

The Fourier transform F(u) 1906 encodes exactly the same information as the spatial-domain image f(x) 1902, except the Fourier transform F(u) 1906 is expressed in terms of amplitude as a function of spatial frequency u, rather than intensity as a function of spatial displacement x. Because of the one-to-one correspondence between the spatial domain and the frequency domain, there are also N₁ points in the frequency domain u.

In general, more computational effort is needed to isolate or remove certain image characteristics in the spatial domain than in the frequency domain. For example, the image data corresponding to the contour, or general outline, of an image appear as distinct, high-frequency components of the Fourier transform in the frequency domain. The method of separating certain components or features of a digital image, such as the contour of an image, whether in the spatial domain or the frequency domain, is referred to as “filtering.” The Fourier transform data associated with the contour of an image can be separated from the rest of the Fourier transform data by multiplying the Fourier transform by a filter function notationally represented by “H(u).” In FIG. 19, the Fourier transform F(u) 1904 is filtered in the frequency domain by multiplied by a filter function H(u) 1908 to give:
G(u)=H(u)F(u) Equation (4)
The resulting function G(u) 1910 is referred to as the “filtered Fourier transform.” The inverse Fourier transform 1912 of the filtered Fourier transform G(u) 1910 produces the desired, filtered, spatial-domain image g(x) 1914, where the inverse Fourier transform 1912 is defined by the following equation:

$${ℱ}^{- 1} [ G ( u ) ] = g ( x ) = \frac{1}{N} {\sum }_{x = 0}^{{N}_{1} - 1} G ( u ) exp ( \frac{2 π i xu}{{N}_{1}} ) \qquad Equation ( 5 )$$

Note that, typically, more computational effort is needed to process a large digital image data set, such as that obtained from reading a multi-pack of microarrays, in the spatial domain than is needed to follow a processing procedure outlined above in relation to FIG. 19.

The number of multiplications and additions required to implement the discrete Fourier Transform given by equation (3) is proportional to N₁². In other words, for each of the N₁ values of u, N₁ complex multiplications of f(x) by the exponential given by:

$$exp ( \frac{- 2 π xui}{{N}_{1}} )$$

are required plus N₁−1 additions. A Fast Fourier Transform (“FFT”) can be implemented to reduce the number of multiplications and additions from N₁² to N₁ log₂ N₁ operations. First, the number of points in the spatial domain x is assumed to be a power of 2:
N₁=2ⁿ
where n is a positive integer. Therefore, N₁ can be expressed as:
N₁=2K
where K is also a positive integer. One of many methods for computing the FFT is presented below, in equations (8)-(14), and is referred to as the “successive doubling method.” The successive doubling method is derived by first substituting equation (7) into equation (3) and separating the odd and even spatial domain elements to give:

$$\begin{matrix}F ( u ) = \frac{1}{2 K} {\sum }_{x = 0}^{2 K - 1} f ( x ) exp ( \frac{- 2 π i x u}{2 K} ) = \frac{1}{2} [ \frac{1}{K} {\sum }_{x = 0}^{K - 1} f ( 2 x ) exp ( \frac{- 2 π i ( 2 x ) u}{2 K} ) + \frac{1}{K} {\sum }_{x = 0}^{K - 1} f ( 2 x + 1 ) exp ( \frac{- 2 π i ( 2 x + 1 ) u}{2 K} ) ] \\ Defining & Equations ( 8 ) \\ {F}_{even} ( u ) = \frac{1}{K} {\sum }_{x = 0}^{K - 1} f ( 2 x ) exp ( \frac{- 2 π i x u}{2 K} ) \\ and & \text{Equation} ( 9 ) \\ {F}_{odd} ( u ) = \frac{1}{K} {\sum }_{x = 0}^{K - 1} f ( 2 x + 1 ) exp ( \frac{- 2 π i x u}{K} ) & \text{Equation} ( 10 )\end{matrix}$$

for u=0,1,2, . . . , K−1, reduces equation (8) to the following:

$$F ( u ) = \frac{1}{2} [ {F}_{even} ( u ) + {F}_{odd} ( u ) exp ( \frac{- 2 π i x u}{2 K} ) ] \qquad Equation ( 11 )$$

The following two equations hold:

$$\begin{matrix}exp ( \frac{- 2 π i x ( u + K ) }{K} ) = exp ( \frac{- 2 π i x u}{K} ) \\ and & Equation ( 12 ) \\ exp ( \frac{- 2 π i x ( u + K ) }{2 K} ) = - exp ( \frac{- 2 π i x u}{2 K} ) & \text{Equation} ( 13 )\end{matrix}$$

Therefore, equations (11)-(13) produce the following result:

$$F ( u + K ) = \frac{1}{2} [ {F}_{even} ( u ) + {F}_{odd} ( u ) exp ( \frac{- 2 π i x u}{2 K} ) ] \qquad Equation ( 14 )$$

Equations (11) and (14) indicate that an N₁-point transformation can be computed by dividing the original expression into two parts. Computing the first half of F(u) requires evaluation of the two (N₁2)−point transformation given by equations (9) and (10). The resulting values of Fₑᵥₑₙ(u) and F_{odd}(u) are then substituted into equation (11) to obtain F(u) for u=0, 1, 2, . . . , (N₁2−1). The other half follows directly from equation (14) without additional transformation evaluations. Note that there exist numerous methods for computing the FFT, and therefore, the present invention is not limited to the successive doubling method described above in relation to equation (7)-(14)

Utilizing the FFT requires the number of points in the spatial domain to conform to equation (6). If the condition presented by equation (6) is not satisfied after the sampling procedure, as described above in relation to FIGS. 14-16, the number of points needed for the FFT, N₂, can be computed using the following equation:
N₂=2^{ceil(log(N}₁)/log(2))
where “ceil” is the integer value just larger than the value determined by:

$$\frac{log ( {N}_{1} )}{log ( 2 )}$$

Therefore, for the projection shown in FIG. 18, N₂ equals 2048 (2¹¹) points in the spatial domain of f(x) in order to enable FFT. An edge fill operation is performed by appending 368 (2048-1680) points having zero spatial-domain image values to the end of the spatial-domain image f(x). This process is referred to as “zero-padding.”

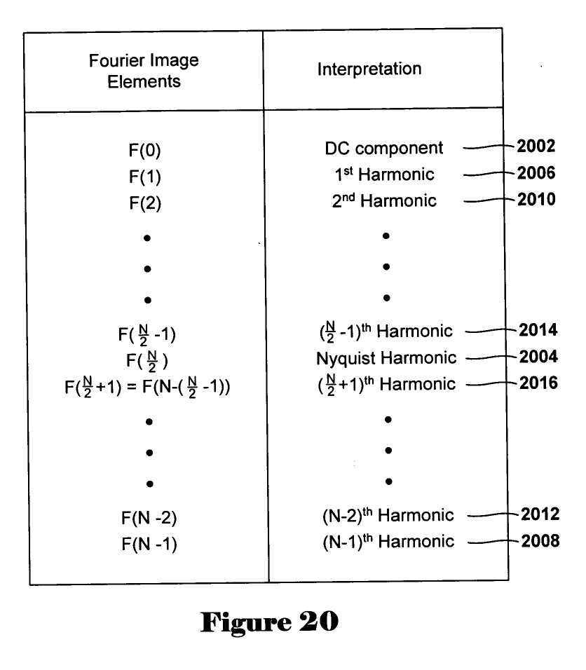

The discrete Fourier transform of any sequence, whether the sequence is real or complex, always results in a complex output of the form:
F(u)=Re{F(u)}+iIm{F(u)}
where Re{F(u)} and Im{F(u)} are the real and imaginary components of the Fourier transform F(u), respectively. FIG. 20 displays the Fourier transform elements and the interpretation of each element. The Fourier transform elements are referred to as “harmonics.” The Fourier transform element F(0) 2002, referred to as the “DC-component,” is real valued and corresponds to the average intensity of the spatial-domain image f(x). An inherent property of the Fourier transform of a real sequence, such as the sequence of elements of the spatial-domain image f(x) 2002 shown in FIG. 18, is that
|F(N₂−u)|²=|F(u)|²

where |F(u)|²=F·F*;
u=1, 2, . . . ,N₂−1; and
F* is the complex conjugate of F
   In other words, the Fourier transform F(u) is conjugate symmetric about the frequency domain points N₂/2, also known as the Nyquist harmonic F(N₂/2) 2004. The magnitude of F(1) 2006 is equal to the magnitude of F(N₂−1) 2008, the magnitude of F(2) 2010 is equal to the magnitude of F(N₂−2) 2012, and the magnitude of F(N₂/2−1) 2014 is equal to the magnitude of F(N₂/2+1) 2016.

Applying the FFT to the multi-pack of microarrays image data yields a representation of the information contained in the image in terms of frequency and phase data. The phase information is typically difficult to display visually, but a power spectrum may be employed as a means of displaying the amplitudes of the frequency component of the Fourier transform. One of many possible methods for computing the power spectrum of the Fourier transform is given by the following expression:
P(u)=|F(u)|²=[Re{F(u)}]²+[Im{F(u)}]²
The contribution to the Fourier transform F(u) made by the contour or general shape of the of the spatial-domain image f(x) are identified in the power spectrum where |F(u)|² has high-frequency amplitude. For example, in FIG. 18, the Fourier transform of the period general form or contour of the intensify bands 1803-1806 and troughs 1808-1810 of the spatial-domain image f(x) appear with high-frequency amplitude in the power spectrum, P(u).

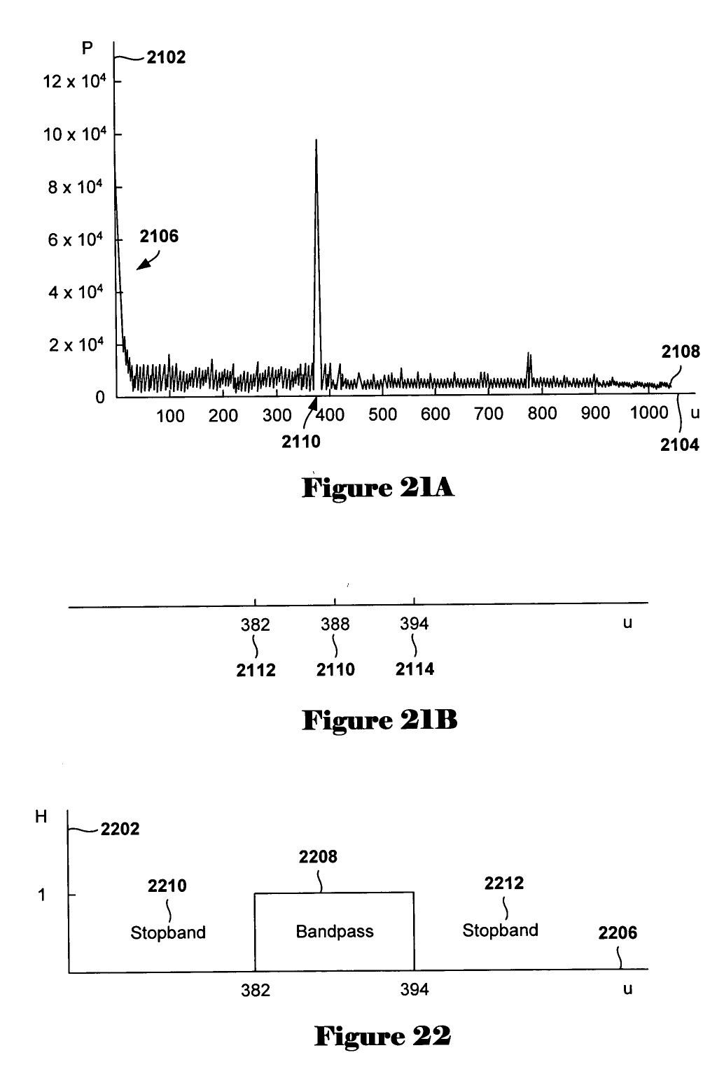

FIGS. 21A-B shows the power spectrum for the spatial-domain image f(x) shown in FIG. 18. FIG. 21A is an illustration of the power spectrum plotted with respected to the P-axis 2102 and the frequency domain represented by the u-axis 2104. Note that only half of the power spectrum is plotted in FIG. 21A, because the power spectrum is symmetric about the Nyquist harmonic in the frequency domain. In FIG. 21A, the peak 2106 is associated with the DC-component 2002 describe above with reference to FIG. 20. The Nyquist harmonic 2004, shown FIG. 20, is represented by the point 2108 at the end of the spectrum. The Fourier transform of the periodic contour of the spatial-domain-image data is identified by the band of frequencies comprising the high-frequency-amplitude spike centered about the frequency 388 Hz (point 2110 in FIG. 21B), which is referred to as “Max_Amplitude.” The endpoints of the band of frequencies, referred to as p₁ and p₂, can be characterized according to the following expressions:
p₁=Max_Amplitude−2 (Bands_{—}x−1) Equation (17)
p₂=Max_Amplitude+2(Bands_{—}x−1) Equation (18)
where Bands_x is the number of intensity projections bands in the spatial domain. For example, the number of intensity projection bands, Bands_x, in the projection 1802, shown in FIG. 18, is “4.” Therefore, the endpoints 2112 and 2114 of the band of frequencies are determined to be 382 and 392 Hz, respectively.

Spatial filtering can be employed to remove the low-amplitude values of |F(u)|² from the image data by designing a filter function that is non-transmitting in the appropriate frequency range. When the image is reconstructed, after having been filtered in the frequency domain, only the image data associated with the contour of the image in the spatial domain remains. Because determining the spacing between individual microarrays is the objective of the present invention, the Fourier transform F(u) is multiplied by a top-hat function:

$$H ( u ) = \{ \begin{matrix}1 & {p}_{1} \leq u \leq {p}_{2} \\ 0 & otherwise\end{matrix} \qquad Equation ( 19 )$$

where p₁ and p₂ are determined according to equations (17) and (18), respectively, in order to select only those amplitudes F(u) in the frequency domain that are associated with the Fourier transform of the contours in the spatial domain. Multiplying the Fourier transform F(u) by the top-hat function H(u) is represented by the equation:
G(u)=H(u)F(u) Equation (20)

for u=0, 1, 2, . . . ,N₂−1

The function defined in equation (19) is referred to as a “bandpass filter.”

FIG. 22 shows the top-hat, bandpass-filter function given in equation (19) plotted with respect to the H-axis 2202 and the u-axis 2206. The output of the product of H(u) with the Fourier transform F(u) consists only of those Fourier transform elements that are within the bandpass 2208. The Fourier transform elements F(u) having frequency domain values in the stopband regions 2210 and 2212 are eliminated from the Fourier transform F(u) leaving the filtered Fourier transform G(u).

The method used to compute the FFT can also be used to compute the inverse FFT. Like the FFT, the inverse FFT is a one-to-one mapping, that maps points in the frequency domain into the spatial domain. The inverse FFT is determined by taking the complex conjugate of equation (3) and dividing both sides by N₁ to give the following equation:

$$\frac{1}{{N}_{1}} f * ( x ) = \frac{1}{{N}_{1}} {\sum }_{u = 0}^{{N}_{1} - 1} F * ( u ) exp ( \frac{- 2 π ixu}{{N}_{1}} ) \qquad Equation ( 21 )$$

The right-hand side of equation (21) is of the form of the Fourier transform given in equation (3). Substituting the complex conjugate of the filtered Fourier transform, G*(u), as described above in relation to equations (6) through (14), gives the quantity g*(x)/N₁. Taking the complex conjugate and multiplying by N₁ produces the desired, filtered, spatial-domain image g(x).

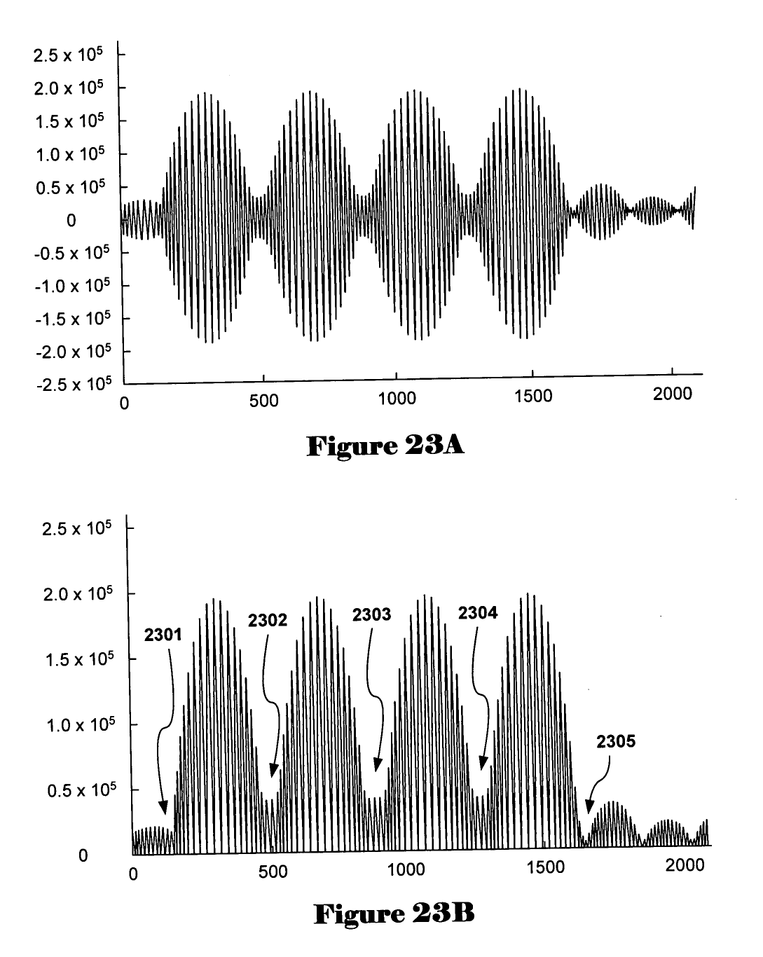

FIGS. 23A-B show the filtered, spatial-domain image g(x) corresponding to the un-filtered, spatial-domain image f(x) 1802 shown in FIG. 18. In FIG. 23A, the inverse FFT, filtered, spatial-domain image g(x), is plotted. FIG. 23B is an illustration of the absolute value of the filtered, spatial-domain image g(x) shown in FIG. 23A. The locations of the microarray boundaries can be estimated from the absolute value of filtered, spatial-domain image g(x). The x coordinates of the boundaries between the microarrays of the 8-pack of microarrays are indicated by the minima 2301-2305.

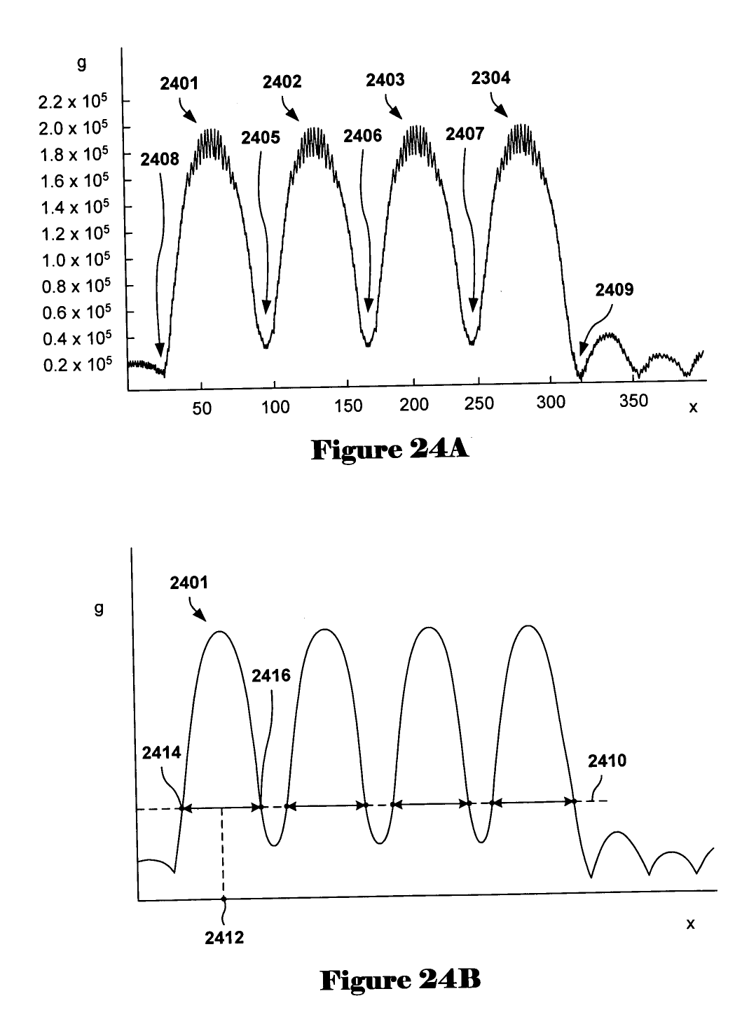

FIGS. 24A-B are illustrations of the peak envelope of the filtered, spatial-domain image g(x) shown in FIG. 23B. First, the filtered, spatial-domain image g(x) shown in FIG. 24A is sampled by convolving g(x) with the sampling function given by:

$$s ( x ) = \{ \begin{matrix}1 & x = nX \\ 0 & otherwise\end{matrix} \\ where n = 0 , 1 , 2 , … , {N}_{3} = ( \frac{{N}_{2}}{X} - 1 ) ; and \\ X = 5.27835 units . \qquad Equation ( 22 )$$

The size of the spatial domain is reduced from 2048 (N₂) points to 388 (N₃) points. FIG. 24B illustrates one of many possible techniques for estimating the spatial domain x coordinates for the peaks 2401-2404 shown in FIG. 24A. Peak finding may be performed by using statistics gathered from the filtered, spatial-domain image g(x). For example, a threshold 2410 is set using statistics of the filtered, spatial-domain image g(x) such as the median. The spatial domain x coordinate 2412 of the first peak 2401 is determined by taking the mid-point between the points 2414 and 2416, which are points where the first rising edge and first falling edge intersect the threshold 2410, respectively. The x coordinate of peaks 2401-2404 of the filtered, spatial-domain image g(x) are 57, 130, 208.75, and 283 and are referred to as the “peakval(i),” where i is the peak index.

The x coordinates of the boundaries are assumed to be midpoints between the peaks 2401-2404. Therefore, the x coordinates of the microarray boundaries can be calculated according to the following equation:

$$boundary ( i ) = \frac{( peakval ( i ) + peakval ( i + 1 ) )}{2} \qquad Equation ( 23 )$$

Using equation (23), the x coordinates of microarray boundaries 2405-2407, shown in FIG. 24B, are calculated to be 93.5, 170.5, and 247, respectively. One of many possible methods for estimating the x coordinates of the outermost microarray boundaries 2408 and 2409 is to assume that peak 2401 is the midpoint of the outermost microarray boundary point 2408 and microarray boundary point 2405, and that peak 2404 is the midpoint between microarray boundary point 2407 and outermost microarray boundary point 2409. Thus, the x coordinates of the outermost microarray boundaries 2408 and 2409 are 20.5 and 319, respectively.

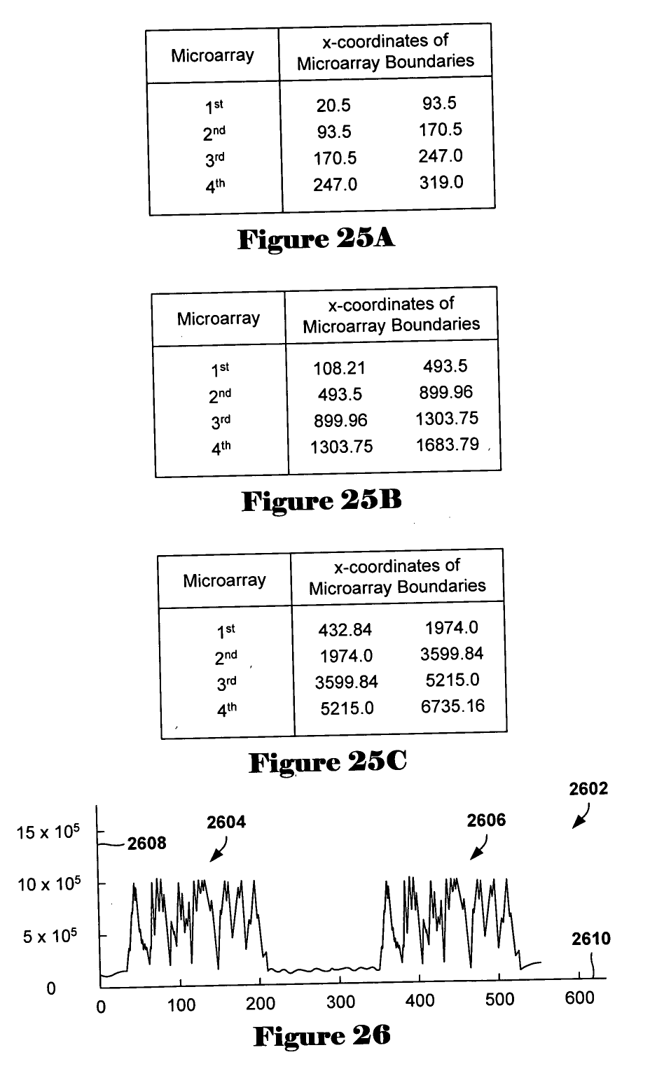

Next, the x coordinates of the microarray boundaries are rescaled to obtain the x coordinates of the microarrays boundaries of the original 8-pack of microarrays shown in FIG. 13. FIG. 25A shows the x coordinates of the microarray boundaries of the example 8-pack of microarrays, determined for N₃ equal to 388 points, as described above in relation to FIGS. 24A-B. In FIG. 25B, the x coordinates are scaled by multiplying by the scale factor N₂N₃ (2048/388). In FIG. 25C, the points that were added to the spatial domain according to equation (15) are subtracted, and the x coordinates are scaled again by multiplying by the factor N/N₁ (6720/1680) to give the x coordinates in the microarray boundaries of the original 8-pack of microarrays.

After the x coordinates of the microarray boundaries of the multi-pack of microarrays have been determined, the image of the multi-pack of microarrays is rotated about an axis perpendicular to the plane of the multi-pack of microarrays and the process related to FIGS. 16-25 is repeated to determine the y coordinates of the microarray boundaries of the multi-pack of microarrays. FIG. 26 illustrates the projection 2602 resulting from projecting the sampled, spatial-domain image of the example 8-pack of microarrays, shown in FIG. 13, after rotating the spatial domain. The projection 2602 is composed of two intensity bands 2604 and 2606 plotted with respect to the intensity-axis 2608 and the y-axis 2610. The intensity bands 2604 and 2606 are the sum of pixels of four microarrays in the example 8-pack of microarrays. The method described above with reference to FIGS. 15-26 is repeated to determine the coordinates of boundaries separating the intensity bands 2604 and 2606.

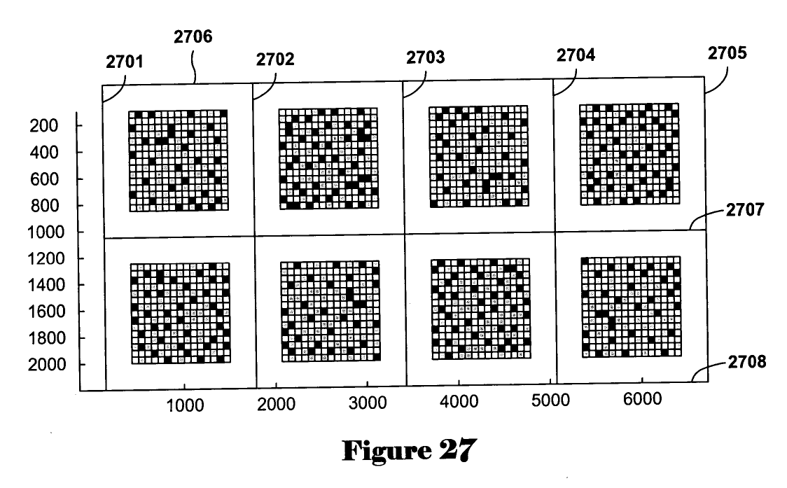

FIG. 27 shows the boundaries separating the individual microarrays within the 8-pack of microarrays shown in FIG. 13. In FIG. 27, vertical lines 2701-2705 and horizontal lines 2706-2708 identify the vertical and horizontal boundaries separating the individual microarrays in the 8-pack of microarrays shown in FIG. 13.

### Implementation

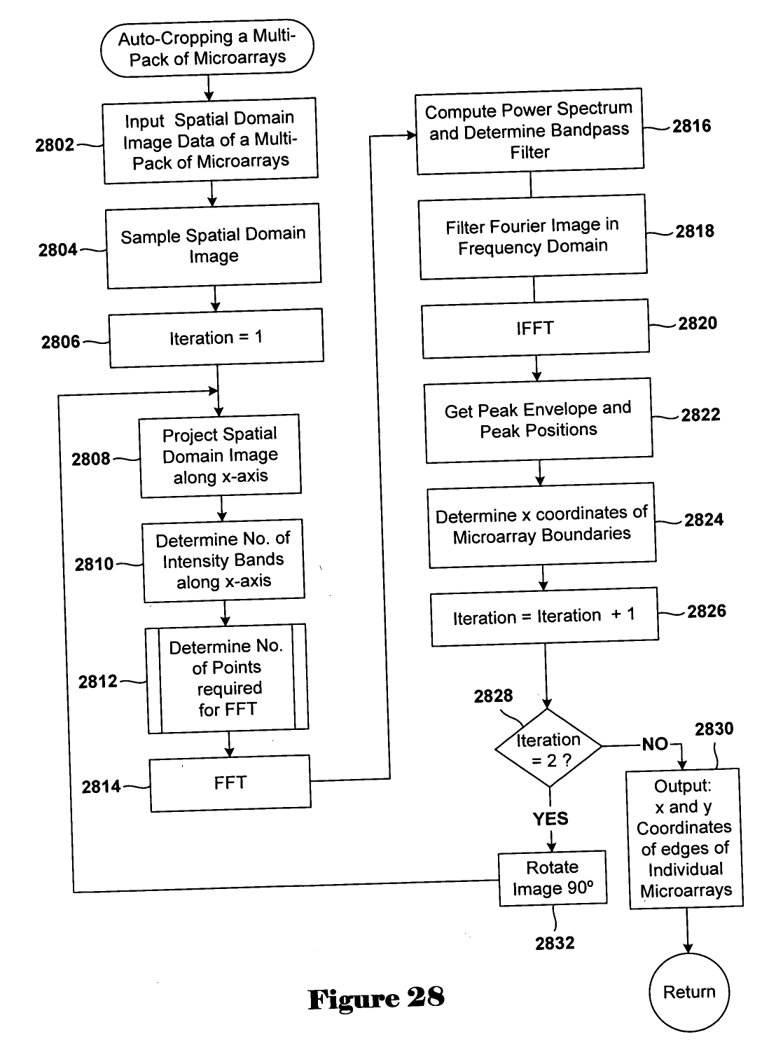

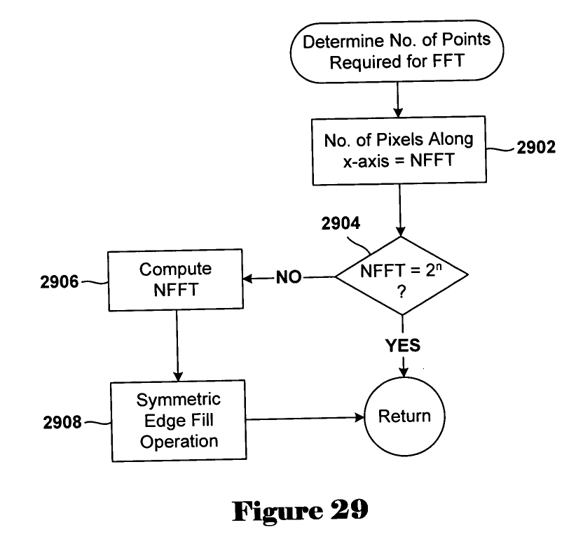

FIGS. 28 and 29 provide a series of control-flow diagrams that describe the method of automated cropping of an image of a multi-pack of microarrays, as described above with reference to FIGS. 13-27. FIG. 28 is a control-flow diagram for the routine “auto-cropping a multi-pack of microarrays.” In step 2802, the spatial-domain image data of a multi-pack of microarrays is provided. In step 2804, the spatial-domain image data is sampled. In step 2806, the variable used to store the number of iterations, “iteration,” is assigned the value “1.” In step 2808, the spatial-domain image data is projected along the x coordinate axis to give a projection, as described above in relation to FIGS. 16-18. In step 2810, the number of intensity bands along the x coordinate axis is determined. In step 2812, the number of points along the x coordinate axis needed for a FFT is determined by calling the routine “determine a number of points required for FFT.” In step 2814, the projection determined in step 2808 is mapped to the frequency domain according to the FFT method described above in relation to equations (6) through (14). In step 2816, the power spectrum is determined as described above in relation to equation (16). In step 2818, the Fourier transform is filtered in the frequency domain according to equations (19) and (20) and as described above in relation to FIGS. 21 and 22. In step 2820, the filtered Fourier transform is mapped back to the spatial domain according to the inverse FFT given in equation (21) to obtain the filtered, spatial-domain image. In step 2822, the peak envelope of the filtered, spatial-domain image is determined, as described above in relation to FIGS. 24A and B. In step 2824, the x coordinates of the microarray boundareis of the multi-pack of microarrays is determined. In step 2826, the variable “iteration” is incremented. In step 2828, if “iteration” equals “2,” then in step 2832, the image of the multi-pack of microarrays is rotated 90 degrees and steps 2808 through 2828 are repeated. In step 2828, if “iteration” does not equal “2,” then in step 2830, the x and y coordinates of the boundaries of the individual microarrays in the multi-pack of microarrays is output.

FIG. 29 is a control-flow diagram for the routine “determine the number of points required for FFT.” In step 2902, the variable “NFFT” is assigned the number of pixels along the x coordinate axis. In step 2904, if there exist an integer n such that “NFFT” is equal to 2ⁿ, then return to the calling routine “auto-cropping a multi-pack of microarrays.” In step 2904, if there does not exist a integer n such that “NFFT” is equal to 2ⁿ, then in step 2906, “NFFT” is assigned an integer value as described above in equation (15). In step 2908, the outside edges of the projection are filled with additional points as described above in relation to equation (15).

Although the present invention has been described in terms of a particular embodiment, it is not intended that the invention be limited to this embodiment. Modifications within the spirit of the invention will be apparent to those skilled in the art. For example, an almost limitless number of different implementations of the many possible embodiments of the method of the present invention can be written in any of many different programming languages, embodied in firmware, embodied in hardware circuitry, or embodied in a combination of one or more of the firmware, hardware, or software, for inclusion in microarray data processing equipment employing a computational processing engine to execute software or firmware instructions encoding techniques of the present invention or including logic circuits that embody both a processing engine and instructions. In alternate embodiments, the edge fill operations can be performed by symmetrically adding points to both the ends of the spatial domain in both the positive and negative x directions. In alternate embodiments, other methods exist for implementing the FFT, and therefore, the present invention is not limited to the FFT successive doubling method described above. In alternate embodiments, other transformation can be employed rather than the Fourier transform, such as the Laplace transform. The method of the present invention is not limited to the multipack of microarrays described above with reference to FIGS. 8, 11, 13, and 27. For example, in alternate embodiments, the method of the present invention can be applied to other arrangements of multiple microarrays, such as microarrays arranged in a near linear fashion.

The foregoing description, for purposes of explanation, used specific nomenclature to provide a thorough understanding of the invention. However, it will be apparent to one skilled in the art that the specific details are not required in order to practice the invention. The foregoing description of specific embodiments of the present invention are presented for purposes of illustration and description. They are not intended to be exhaustive or to limit the invention to the precise forms disclosed. Obviously many modifications and variations are possible in view of the above teachings. The embodiments are shown and described in order to best explain the of the invention and its practical applications, to thereby enable others skilled in the art to best utilize the invention and various embodiments with various modifications as are suited to the particular use contemplated. It is intended that the scope of the invention be defined by the following claims and their equivalents:

What is claimed is:

1. A method for cropping a digital image of multiple individual microarrays, the method comprising:
projecting the digital image along a first axis to produce a first axis projection of a first set of one or more intensity bands;
based on the first axis projection, determining coordinates of boundaries along the first axis projection that separate the one or more intensity bands; and
based on the coordinates of boundaries along the first axis that separate the one or more intensity bands, identifying a location which separates the one or more individual microarrays.

2. The method of claim 1 wherein identifying the location which separates the one or more individual microarrays further includes identifying one or more spacings between intensity bands which are greater than smaller spacings within the individual microarrays.

3. The method of claim 1 wherein projecting the digital image along an axis further includes summing columns of pixel intensities to form the projection along the first axis.

4. The method of claim 1 wherein determining the coordinates of boundaries separating one or more intensity bands further includes:
transforming the projected digital image into a transform in a frequency domain;
filtering the transform in the frequency domain; and
inverse transforming the filtered transform into a filtered, projected digital image.

5. The method of claim 4 wherein filtering the transform in the frequency domain further includes passing a high frequency band.

6. The method of claim 4 further including:
determining the number of intensity bands along the coordinate axes.

7. The method of claim 4 wherein transforming the projected digital image further includes employing a Fourier transform.

8. The method of claim 7 further including:
adding more pixel coordinates to the first axis if the number of points along the first axis does not equal 2ⁿ for some positive integer value of n.

9. The method of claim 4 wherein filtering the transform in the frequency domain further includes:
determining a power spectrum;
based on the power spectrum, determining a filter function; and
multiplying the transform by the filter function.

10. The method of claim 1 further including:
rotating the digital image of multiple individual microarrays about an axis perpendicular to the plane of the digital image and repeating the method of claim 1.

11. Transferring results produced by a microarray reader or microarray data processing program employing the method of claim 1 stored in a computer-readable medium to an intercommunicating entity.

12. Transferring results produced by a microarray reader or microarray data processing program employing the method of claim 1 to an intercommunicating entity via electronic signals.

13. A computer program including an implementation of the method of claim 1 stored in a computer-readable medium.

14. A method comprising forwarding data produced by employing the method of claim 1 to a remote location.

15. A method comprising receiving data produced by employing the method of claim 1 from a remote location.

16. A microarray reader that employs the method of claim 1 to crop the digital image of multiple individual microarrays.

17. A system crops digital image of multiple individual microarrays, the system comprising:
a computer processor;
a communications medium by which microarray data are received by the molecular-array-data processing system;
a program, stored in the one or more memory components and executed by the computer processor that projects the digital image along a first axis to produce a first axis projection of a first set of one or more intensity bands; based on the first axis projection, determines coordinates of boundaries along the first axis projection that separate the one or more intensity bands; and based on the coordinates of boundaries along the first axis that separate the one or more intensity bands, identifies a location which separates the one or more individual microarrays.

18. The system of claim 17 wherein crops the background-intensity component further includes:
computes a transform of the projected digital image;
filters the transform in the frequency domain; and
computes an inverse transform of the filtered transform to give a filtered, projected digital image.

19. The system of claim 17 wherein filters the transform in the frequency domain further includes computes a power spectrum and multiplies the transform by a filter function.

20. The system of claim 17 further includes rotates the digital image data about an axis perpendicular to the plane of the digital image of multiple individual microarrays and repeats the method of claim 17.
